(19) United States
(12) Patent Application Publication
(10) Pub. No.: US 2012/0095337 A1
(43) Pub. Date: Apr. 19, 2012

(54) SYSTEMS AND METHODS TO IMPROVE ULTRASOUND BEAMFORMING

(76) Inventor: Radu Alexandru, Cheshire, CT (US)

(21) Appl. No.: 13/273,650

(22) Filed: Oct. 14, 2011

Related U.S. Application Data
(60) Provisional application No. 61/393,053, filed on Oct. 14, 2010.

(51) Int. Cl. A61B 8/00

(52) U.S. Cl. 600/442

(57) ABSTRACT

Systems and methods are disclosed to receive a plurality of samples of an ultrasound beam, each of the plurality of samples associated with a tissue depth, determine, for each of a plurality of candidate depths, a difference between the sum of each of the plurality of samples which is associated with a tissue depth from the candidate depth to a predetermined distance below the candidate depth, and the sum of each of the plurality of samples which is associated with a tissue depth from the candidate depth to the predetermined distance above the candidate depth, determine the largest determined difference, and determine the one of the plurality of candidate depths associated with the largest determined difference.

### CROSS-REFERENCE TO RELATED APPLICATION

The present application claims priority to U.S. Provisional Patent Application Ser. No. 61/393,053, filed on Oct. 14, 2010 and entitled “Method To Improve Ultrasound Beamforming”, the contents of which are incorporated herein by reference for all purposes.

### BACKGROUND

1. Field

The present invention relates generally to ultrasound imaging. More specifically, some embodiments of the invention relate to systems and methods for focusing ultrasound beams.

2. Description

In ultrasound imaging, a transducer is used to transmit ultrasound beams into a medium such as a human body. The transmitted beams are reflected from various discontinuities in a region of the human body, thereby creating echo waves, some of which return to the transducer. The transducer converts the received waves into electrical signals which undergo a number of signal and image processing steps and are eventually transformed into an image which can be displayed or printed for examination.

Often the transducer consists of a number of small elements arranged along a straight line (linear array) or along an arc (convex array). For each beam, a subset of the elements is used to form the transmit and receive apertures. The transmitted beam is focused by delaying the electrical signal applied to each element in the transmit aperture such that the ultrasound waves emanating from all elements arrive at the same time (in phase) at the desired focus point. Likewise the receive signal from each element in the receive aperture is delayed such that the delayed signals returning from the points of interest are in phase. Therefore, when the delayed receive signals are summed, the signals representative of the points of interest add constructively, enhancing each other, while the signals representative of other points in the body add destructively, canceling each other, resulting in a signal which is primarily representative of the points of interest. The processes of focusing the transmit and receive beams described above are known as transmit and receive beamforming respectively, and the devices used to carry out these processes are called transmit and receive beamformers respectively.

The time delays used in beamforming are calculated based on the distance from the elements to the focus points of interest and the ultrasound velocity in the medium. Traditionally in medical ultrasound imaging, the delay calculations are based on a simplified model which assumes a uniform sound velocity (typically 1540 m/s) and straight line propagation. However it is known that various types of tissue exhibit different sound propagation velocities. Therefore the delays calculated with the traditional uniform velocity assumption may be incorrect and result in a type of focusing error known as phase aberration.

There is a large body of research addressing the problem of correcting phase aberration. Currently, a practical general method to correct all types of phase aberration encountered in medical ultrasound imaging is not available. However, approximate correction methods have been developed for certain situations. One such method models the body as consisting primarily of 2 layers: a superficial layer of fat (sound velocity 1450 m/s) over a layer of other tissues (average sound velocity 1540 m/s). The fat/tissue interface is approximately parallel to the transducer surface. When this model is valid the delays may be calculated using different sound velocities for the portions of the element-to-point propagation paths situated in the 2 layers.

To calculate the length of the propagation path in the first (fat) and second (tissue) layer, the depth of the fat/tissue interface has to be known. In many ultrasound images the subcutaneous fat appears as a dark region with its borders approximately parallel to the transducer surface and situated between the bright transducer/skin line, and the bright muscle region situated underneath the fat layer. This suggests using conventional image processing algorithms such as edge detection and line fitting to determine the position of the fat/tissue border. However, certain characteristics of ultrasound images make this straightforward approach impractical. First, the fat layer may not appear uniformly dark, but rather as a dark background containing bright lines representative of connective tissue or other types of tissue situated in the fat layer. Second, some portions of the image may be dark far further than the fat layer depth because of poor contact between portions of the transducer and the body. Third, ultrasound images have a grainy appearance (speckle) which may make edge detection difficult. Fourth, the assumption of parallelism of the fat/tissue border to the transducer surface is only an approximation, and when the actual border is not parallel to the transducer, an equivalent parallel border must be found. This may be difficult especially for curved array transducers where the equivalent border is an arc of circle. Methods are known to deal with these and other difficulties, but they usually impose a significant computational burden on the ultrasound system. There is a need for a simple method to estimate the depth of the fat/tissue border.

### SUMMARY

According to some embodiments, the fat/tissue border is estimated using detected and processed beam envelope signals (before scan conversion). For each of a plurality of candidate depths, a difference is calculated between the sum of all samples in an area extending a certain distance from the candidate depth to a predetermined distance below the candidate depth, and the sum of envelopes of all samples in the area extending from the candidate depth to the predetermined distance above the candidate depth. The candidate depth for which the calculated difference is the largest is selected as the depth of the fat/tissue border.

In some embodiments the difference is calculated recursively. That is, the difference at depth i+1 is calculated from the difference at depth i using only two additions and one subtraction.

### BRIEF DESCRIPTION OF THE DRAWINGS

FIG. 1 shows a block diagram of a conventional ultrasound scanner (prior art).

FIG. 2 shows a linear array imaging geometry for a homogeneous model.

FIG. 3 shows a linear array imaging geometry for a 2-layer model.

FIG. 4a shows an idealized beam envelope signal.

FIG. 4b shows a difference signal corresponding to FIG. 4a.

FIG. 5a shows a more realistic beam envelope signal.

FIG. 5b shows a difference signal corresponding to FIG. 5a.

FIG. 6 shows a convex array imaging geometry for a 2-layer model.

FIG. 7 shows a block diagram of an ultrasound scanner according to some embodiments of the invention.

### DETAILED DESCRIPTION

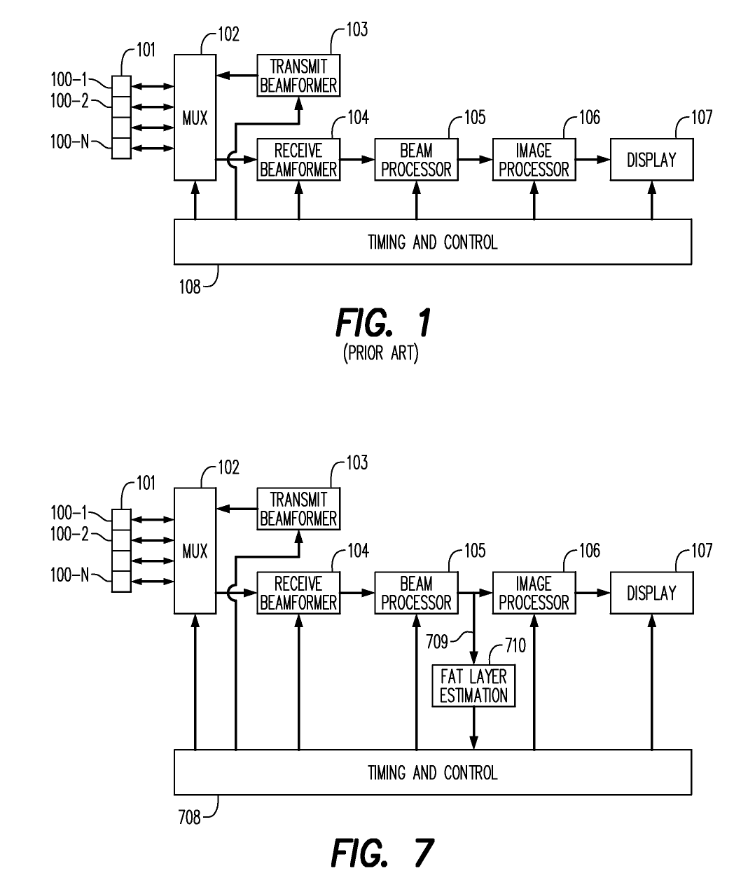

FIG. 1 shows a simplified block diagram of a conventional ultrasound scanner. Various implementations may contain additional blocks or have a different organization but the main functions depicted in the figure and described in the following text are representative of the structure and operation of most ultrasound scanners.

Transducer 101 consisting of one or more elements 101-1, 101-2, etc . . . is used first to transmit and then to receive ultrasound waves and to convert them from or to electrical signals respectively. Multiplexer 102 selects the subset of elements used to transmit/receive at a certain time and also provides isolation between the outputs of the transmit beamformer and the inputs of the receive beamformer. In some implementations the isolation function is implemented separately from the multiplexer. Transmit beamformer 103 generates electrical signals and applies them to the elements of the transducer 101 via the multiplexer 102. The electrical signals applied to various elements may have different delays and amplitudes calculated such as to obtain the desired transmit beam pattern (typically a beam focused at a certain point in the body). The transmit delays may be calculated in the transmit beamformer based on parameters downloaded from the timing and control block 108, or they may be calculated in the timing and control block 108 and then downloaded to the transmit beamformer 103.

Receive beamformer 104 amplifies, filters, delays, weighs (apodizes) and adds the echo signals received from the transducer 101 via multiplexer 102 forming a signal representative of the reflectivity of a sequence of points in the body. In some systems multiple receive beams may be generated in parallel from the signals received from one transmit event. The receive delays may be calculated in the receive beamformer 104 based on parameters downloaded from the timing and control block 108, or they may be calculated in the timing and control block 108 and then downloaded to the receive beamformer 104.

The beam processor 105 receives the beam signal from the receive beamformer 104 and processes it according to the desired imaging mode, e.g. B-mode, Doppler, Color Flow, Elasticity, etc. . . . In the present invention we are interested in B-mode, for which the beam signal's envelope is obtained and in addition some other processing steps such as filtering, amplification, log conversion, etc . . . may be applied.

The image processor 106 receives the mode-specific processed beams from the beam processor 105 and uses them to create an image which, in the B-mode case, is representative of the cross-sectional anatomical structure of the body under examination. The image is then displayed on a display device such as a cathode ray tube or a liquid crystal panel and may also be stored, printed or sent to another device via a network (functions not shown in the figure). All the blocks are synchronized and controlled by timing and control block 108 which receives commands via a user interface input (not shown) and programs the timing and operation of the other blocks accordingly.

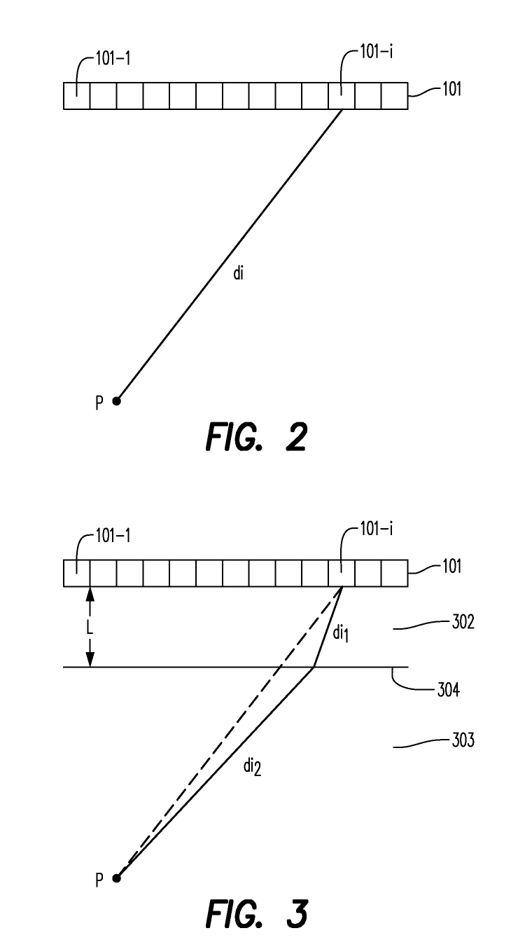

In the prior art system of FIG. 1, the transmit and receive delays are calculated assuming a homogeneous medium of constant sound velocity. FIG. 2 shows the linear array imaging geometry for the homogeneous medium model. Transducer 101 consists of elements 101-1 . . . 101-N arranged along a straight line. The beamforming delays for element 101-i are calculated based on the propagation time tᵢ obtained from the distance dᵢ from the center of the element to the focal point of interest P, and the assumed sound velocity v0, typically 1540 m/s:

$${t}_{i} = \frac{{d}_{i}}{v 0} \qquad ( 1 )$$

where the distance dᵢ is calculated from simple geometrical considerations well-known to one of ordinary skill in the art.

As has already been mentioned, in reality the body is not homogeneous with respect to sound velocity and may sometimes be modeled as consisting of 2 layers: a fat layer of sound velocity v1 (typically 1450 m/s) and a tissue layer of sound velocity v2 (typically 1540 m/s). FIG. 3 shows this situation for a linear array. The transducer 101 has the same geometry as described above for the homogeneous case but the image area has a layer of fat 302, of thickness L, followed by a tissue layer 303, the two layers separated by a boundary 304 running approximately parallel to the surface of transducer 101. Other layers are present as well, such as the skin, but these are thin and may be neglected in a first approximation. The sound propagation time from element 201-i to the focal point P is now calculated as the sum of the propagation times in the 2 layers:

$${t}_{i} = \frac{{d}_{i 1}}{v 1} + \frac{{d}_{i 2}}{v 2} \qquad ( 2 )$$

The 2 distances dᵢ₁ and dᵢ₂ may be calculated with or without consideration of the refraction effect as is known to those in the art. Note that FIG. 3 shows the case in which the refraction effect is considered, that is the propagation is not along a straight line but rather the path in the fat layer is shortened and the path in the tissue layer is lengthened relative to the straight line such as to minimize the total propagation time. Whether the refraction effect is considered or not, calculation of the propagation time requires knowledge of the fat layer thickness L.

Typically, the ultrasound reflections from the fat layer 302 have much lower intensity than the reflections from the muscle usually situated in the tissue layer 303 adjacent to the layer interface 304. Some embodiments of the present invention detect the depth L of the fat/tissue interface 304 as the depth of maximum low-to-high intensity transition as we move from the transducer surface towards deeper depth. To facilitate the computations, the calculations are performed in beam space, that is on the envelope outputs of the beam processor 105.

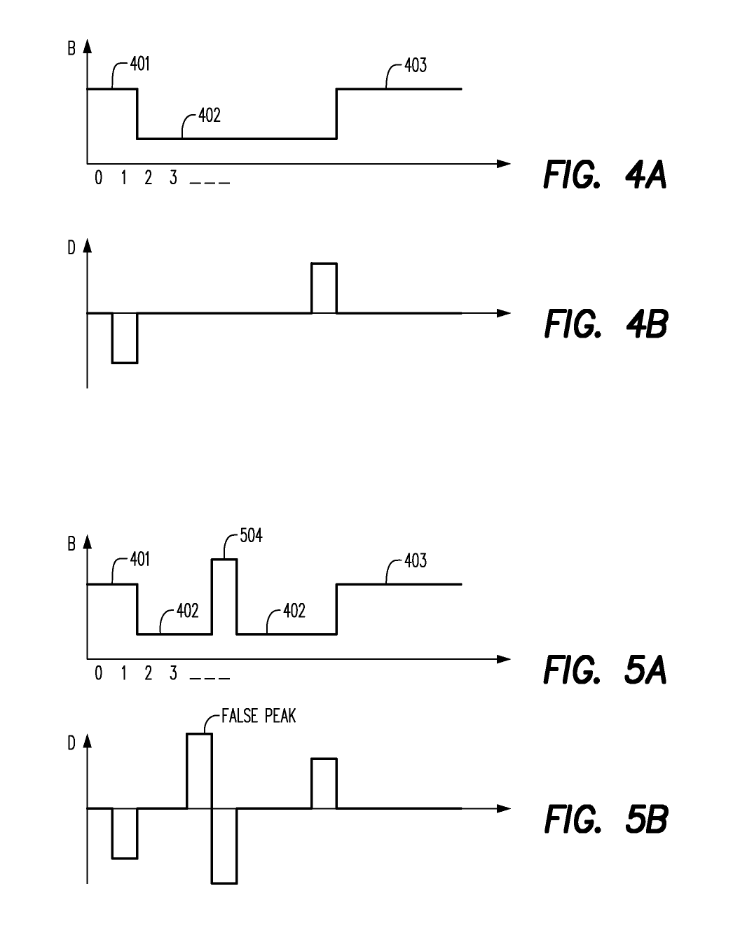

Consider first the idealized detected beam of FIG. 4a. In FIG. 4a, the vertical axis represents the magnitude of the beam's envelope and the horizontal axis represents the depth expressed in number of samples. The graph in FIG. 4a has a narrow high-intensity region 401 at the skin line followed by a low intensity region 402 in the fat layer, followed by another high intensity region 403 in the tissue layer. If at each depth i starting from 0 and up to a preselected maximum depth we calculate the difference

Dᵢ=Bᵢ₊₁−Bᵢ (3)

we obtain the difference graph of FIG. 4b, which has a maximum value at the border of regions 402 and 403. Thus, the sample number iₘₐₓ at which Dᵢ has the maximum value indicates the depth L of the fat/tissue border.

FIG. 5a shows the more realistic case where some strong reflection 504 occurs in the fat layer due for example to the presence of connective tissue membranes. If we use the differentiation method described above then a peak occurs at the strong reflector position which may be mistakenly interpreted as the fat/tissue interface, as illustrated in FIG. 5b. To solve this problem the sample differences of (3) are replaced by differences of sums over N samples before and N samples after each depth position i:

$${D}_{i} = {\sum }_{j = 0}^{N - 1} {B}_{i + N - j} - {\sum }_{j = 0}^{N - 1} {B}_{i - j} \qquad ( 4 )$$

With a proper choice of N, an averaging effect occurs, reducing the influence of isolated bright samples in the fat layer. N should be large enough to significantly reduce the influence the isolated bright samples, but should not exceed the minimum expected thickness of the fat layer. It was found experimentally that a value of N corresponding to a distance of a few mm provides good performance in most cases.

In practical situations, the depth of the fat/tissue boundary may differ from beam to beam and a value representative of the boundary depth on most beams must be found. One approach is to find the boundary depth on each beam by looking for the maximum of the difference (4), and then to apply averaging, line fitting or some other method known in the art to select a representative fat/tissue boundary depth. However this approach may be computationally expensive and prone to errors. In some embodiments, the sums used to calculate the differences (4) are extended over a number of beams:

$${D}_{i} = {\sum }_{k = 1}^{M} {\sum }_{j = 0}^{N - 1} {B}_{k , i + N - j} - {\sum }_{k = 1}^{M} {\sum }_{j = 0}^{N - 1} {B}_{k , i - j} \qquad ( 5 )$$

where M is the number of beams, which may be from 1 to all the beams in the image. This has an averaging effect and provides an estimate approximately valid for all beams.

According to the considerations described above, the algorithm for detecting the fat/tissue boundary may be summarized as follows:

ALGORITHM 1

initialize Dₘₐₓ = 0, iₘₐₓ = N − 1
(where Dₘₐₓ, iₘₐₓ are the value and position of the maximum of the
difference (5) )
for i = N − 1 to Lmax

calculate first and second double sums of (5)
calculate the difference Dᵢ of (5)
if Dᵢ > Dₘₐₓ

update Dₘₐₓ = Dᵢ, iₘₐₓ = i

end
L = iₘₐₓ

Lmax is the maximum expected depth of the fat/tissue boundary, typically a few cm or a few thousand samples. Note that the search does not start from 0 but from a sample corresponding to the number of samples in the double sums of (5).

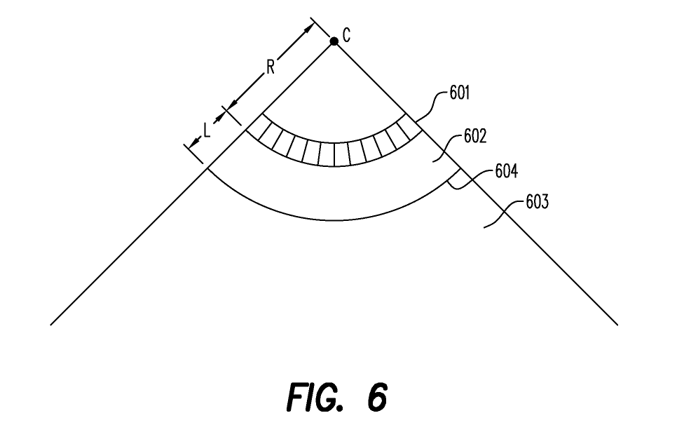

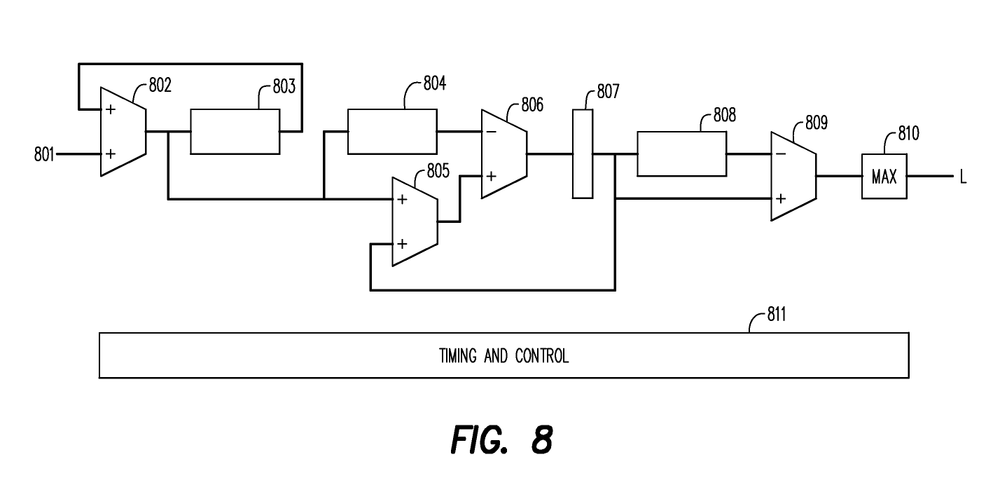

The algorithm for estimating the thickness of the depth of the fat/tissue interface has been described for a linear array transducer. FIG. 6 shows a convex (curved) transducer of radius R imaging a medium consisting of the sector-shaped fat layer 602 and the tissue layer 603, the two layers separated by a border 604 having the shape of an arc of circle with the same center C as the transducer and radius R+L. In this case, the beam intensity graphs will preserve the character of the graphs of FIG. 4a or FIG. 5a, in particular the fat layer depth will be approximately the same (L) on all beams, therefore all the considerations leading to algorithm 1 are applicable to the convex array as well.

Algorithm 1 may be computationally expensive because of the large number of additions needed to calculate the double sums of formula (5). The computational burden may be reduced by calculating formula (5) recursively. We define Hᵢ as the sum of samples across the beams at sample depth i:

$${H}_{i} = {\sum }_{k = 1}^{M} {B}_{k , i} \qquad ( 6 )$$

and observe that the difference (5) at depth i+1 may be obtained from the difference at depth i as follows:

$$\begin{matrix}{D}_{i + 1} = {\sum }_{k = 1}^{M} {\sum }_{j = 0}^{N - 1} {B}_{k , i + 1 + N - j} - {\sum }_{k = 1}^{M} {\sum }_{j = 0}^{N - 1} {B}_{k , i + 1 - j} \\ = {\sum }_{j = 0}^{N - 1} {H}_{i + 1 + N - j} - {\sum }_{j = 0}^{N - 1} {H}_{i + 1 - j} \\ = {\sum }_{j = 0}^{N - 1} {H}_{i + N - j} - {H}_{i + 1} + {H}_{i + 1 + N} - {\sum }_{j = 0}^{N - 1} {H}_{i - j} + {H}_{i - N + 1} - {H}_{i + 1} \\ = {D}_{i} + {H}_{i - N + 1} - 2 \times {H}_{i + 1} + {H}_{i + 1 + N}\end{matrix} \qquad ( 7 )$$

Accordingly, the sums across beams Hᵢ are calculated first, then the first difference D_{N-i} is calculated by subtracting the first N Hᵢ sums and adding the next N, after which successive Dᵢ differences are calculated recursively using formula (7).

ALGORITHM 2

```
initialize Dₘₐₓ = 0, iₘₐₓ = N − 1
for i = 0 to Lmax + N
  $calculate sums {H}_{i} = {\sum }_{k = 1}^{M} {B}_{k , i}$
$for i = N - 1 calculate first difference {D}_{i} = {\sum }_{j = 0}^{N - 1} {H}_{i + N - j} - {\sum }_{j = 0}^{N - 1} {H}_{i - j}$
if Dᵢ > Dₘₐₓ
  update Dₘₐₓ = Dᵢ, iₘₐₓ = i
for i = N to Lmax
  calculate difference Dᵢ = Dᵢ₋₁ + H_{i-N} − 2 × Hᵢ + H_{i+N}
  if Dᵢ > Dₘₐₓ
    update Dₘₐₓ = Dᵢ, iₘₐₓ = i
end
L = iₘₐₓ
```

FIG. 8 shows a block diagram of an algorithm equivalent to algorithms 1 and 2, but optimized for hardware implementation. The circuit calculates the double sums of formula (5) recursively:

$$\begin{matrix}{S}_{i} = {\sum }_{k = 1}^{M} {\sum }_{j = 0}^{N - 1} {B}_{k , i - j} \\ = {\sum }_{j = 0}^{N - 1} {H}_{i , j} \\ = {\sum }_{j = 1}^{N} {H}_{i - j} - {H}_{i - N} + {H}_{i} \\ = {S}_{i - 1} - {H}_{i - N} + {H}_{i}\end{matrix} \qquad ( 8 )$$

and uses each double sum twice, once with positive and once with negative sign, thus reducing the number of computations. Data arrives at the input 801 in beam and sample sequence, that is first sample 0 of beam 1, then sample 1 of beam 1, etc . . . until all samples of beam 1 arrive, then sample 0 of beam 2, sample 1 of beam 2, and so on until all samples of all beam are fed to the circuit. Adder 802 and memory 803 (which may be a FIFO or a shift register) are used to calculate the sums Hᵢ. Dual port memory 803 has Lmax+N+1 locations, each holding a sum Hᵢ. While the samples of the first beam are fed to input 801, timing and control block 811 controls the adder 802 to pass the unmodified samples B_{1,i} to the input of memory 803, and controls the memory 803 to write the samples to the respective sums Hᵢ, initializing them. When samples B_{k,i} from subsequent beams arrive, the adder 802 is controlled to add them to the respective sums Hᵢ read from the memory 803 and the memory 803 is controlled to write the updated sum to its storage location. Memory 804, adder 805, subtractor 806 and register 807 are used for the recursive calculation of the double sums Sᵢ. Dual-port memory 804 (which may be a FIFO or a shift register) operates as a delay line with delay N. After sample 0 of the last beam M arrives at the input 801, the first complete sum H₀ becomes available at the output of adder 802 and is written to memory 804. At the same time adder 805 and subtractor 806 are controlled to pass the sum H₀ unmodified to register 807 which is thus initialized with the first term of the first sum S₀. After sample 1 of last beam M arrives at the input 801, the second complete sum H₁ becomes available at the output of adder 802 and is written to memory 804. At the same time adder 805 is controlled to add the new sum to the output of register 807 and subtractor 806 is controlled to pass this partial sum unmodified to register 807. The last described operation is repeated until sample N−1 of beam M arrives and the first sum S₀ is completed. After that, when each new sum Hᵢ is calculated it is added by adder 805 to the output of register 807 and is written to memory 804 at the location of the old H_{i-N} sum which is read from memory 804 and subtracted from the output of adder 805, thus producing the new sum Sᵢ according to the recursive formula (8).

Memory 808 and subtractor 809 are used to calculate the difference Dᵢ=S_{i+N}−Sᵢ. Dual port memory 808 (which may be a FIFO or a shift register) operates as a delay line of delay N for the sums Sᵢ. When sum S_{N} becomes available at the output of register 807, sum S₀ is available at the output of memory 808 and the subtractor 809 may calculate the first difference D₀. Subsequently a new difference is calculated at each sample clock. The differences Dᵢ are fed to the maximum value detector 810 which then produces the depth estimate L. Timing and control block 812 controls the sequence of operations for all arithmetic and memory units of the circuit. It will be obvious to in the art that registers (not shown) must be used between certain blocks in the circuit in order to meet timing requirements.

FIG. 7 shows a block diagram of an ultrasound scanner incorporating some of the teachings herein. The scanner of FIG. 7 provides all the functionality of the prior art scanner of FIG. 1 plus the following: a data path 709 from the output of the beam processor brings beam envelope signals to the fat layer estimation block 710. Fat layer estimation block 710 implements algorithm 1 or a modified version of it, such as algorithm 2, to estimate the depth L of the fat/tissue interface. Fat layer estimation block 710 may be a hardware circuit such as the one shown in FIG. 8, or a processor or DSP programmed to execute algorithm 1 or its equivalents, or a program code executed by one of the processors already present in the timing and control block 708.

The fat layer estimation process may be activated upon an operator command, for example at the beginning of a scanning session, or it may run periodically in each frame or once every K frames, with the estimate obtained in one frame being used to calculate delays for all subsequent frames until a new estimate is produced. The delay calculation or delay parameter calculation algorithm in the timing and control unit 708 is modified to use the fat/tissue interface depth for calculating delays according to formula (2) with or without consideration of the refraction effect.

FIGS. 7 and 8 represent logical architectures according to some embodiments, and actual implementations may include more or different elements arranged in other manners. Other topologies may be used in conjunction with other embodiments. Moreover, each element may be implemented by any number of computing devices in communication with one another via any number of other public and/or private networks. Two or more of such computing devices may be located remote from one another and may communicate with one another via any known manner of network(s) and/or a dedicated connection. The system may comprise any number of hardware and/or software elements suitable to provide the functions described herein as well as any other functions. For example, any computing device used in an implementation of the some embodiments may include a processor to execute program code such that the computing device operates as described herein.

All systems and processes discussed herein may be embodied in program code stored on one or more non-transitory computer-readable media. Such media may include, for example, a floppy disk, a CD-ROM, a DVD-ROM, a Blu-ray disk, a Flash drive, magnetic tape, and solid state Random Access Memory (RAM) or Read Only Memory (ROM) storage units. Embodiments are therefore not limited to any specific combination of hardware and software.

One or more embodiments have been described. Nevertheless, various modifications will be apparent to those in the art.

What is claimed is:

1. A method comprising:
receiving a plurality of samples of an ultrasound beam from a transducer, each of the plurality of samples associated with a tissue depth;
for each of a plurality of candidate depths, determining a difference between the sum of each of the plurality of samples which is associated with a tissue depth from the candidate depth to a predetermined distance below the candidate depth, and the sum of each of the plurality of samples which is associated with a tissue depth from the candidate depth to the predetermined distance above the candidate depth;
determining the largest determined difference;
determining the one of the plurality of candidate depths associated with the largest determined difference;
calculating a time delay based on the candidate depth associated with the largest determined difference; and
transmitting a second ultrasound beam based on the calculated time delay.

2. A method according to claim 1, wherein determining the difference for each of the plurality of candidate depths comprises determining, over N samples above and N samples below, for each candidate depth i:
$${D}_{i} = {\sum }_{j = 0}^{N - 1} {B}_{i + N - j} - {\sum }_{j = 0}^{N - 1} {B}_{i - j}$$
where Dᵢ is the difference determined for candidate depth i and Bᵢ is a beam sample at candidate depth i.

3. A method according to claim 1, wherein determining the difference for each of the plurality of candidate depths comprises determining, over N samples above and N samples below, for each candidate depth i, and for each of a number of ultrasound beams M:
$${D}_{i} = {\sum }_{k = 1}^{M} {\sum }_{j = 0}^{N - 1} {B}_{k , i + N - j} - {\sum }_{k = 1}^{M} {\sum }_{j = 0}^{N - 1} {B}_{k , i - j}$$

4. A method according to claim 3, wherein determining the difference for each of the plurality of candidate depths comprises determining the difference at depth i+1 from the difference at depth i as:
$$\begin{matrix}{D}_{i + 1} = {\sum }_{k = 1}^{M} {\sum }_{j = 0}^{N - 1} {B}_{k , i + 1 + N - j} - {\sum }_{k = 1}^{M} {\sum }_{j = 0}^{N - 1} {B}_{k , i + 1 - j} \\ = {\sum }_{j = 0}^{N - 1} {H}_{i + 1 + N - j} - {\sum }_{j = 0}^{N - 1} {H}_{i + 1 - j} \\ = {\sum }_{j = 0}^{N - 1} {H}_{i + N - j} - {H}_{i + 1} + {H}_{i + 1 + N} - {\sum }_{j = 0}^{N - 1} {H}_{i - j} + {H}_{i - N + 1} - {H}_{i + 1} \\ = {D}_{i} + {H}_{i - N + 1} - 2 \times {H}_{i + 1} + {H}_{i + 1 + N}\end{matrix}$$
where Dᵢ₊₁ is the difference determined for candidate depth i+1, and Hᵢ is the sum of samples across the beams at sample depth i:
$${H}_{i} = {\sum }_{k = 1}^{M} {B}_{k , i} .$$

5. A non-transitory medium storing processor-executable program code, the program code executable by a device to:
receive a plurality of samples of an ultrasound beam from a transducer, each of the plurality of samples associated with a tissue depth;
for each of a plurality of candidate depths, determine a difference between the sum of each of the plurality of samples which is associated with a tissue depth from the candidate depth to a predetermined distance below the candidate depth, and the sum of each of the plurality of samples which is associated with a tissue depth from the candidate depth to the predetermined distance above the candidate depth;
determine the largest determined difference;
determine the one of the plurality of candidate depths associated with the largest determined difference;
calculate a time delay based on the candidate depth associated with the largest determined difference; and
transmit a second ultrasound beam based on the calculated time delay.

6. A medium according to claim 5, wherein determination of the difference for each of the plurality of candidate depths comprises determination of, over N samples above and N samples below, for each candidate depth i:
$${D}_{i} = {\sum }_{j = 0}^{N - 1} {B}_{i + N - j} - {\sum }_{j = 0}^{N - 1} {B}_{i - j}$$
where Dᵢ is the difference determined for candidate depth i and Bᵢ is a beam sample at candidate depth i.

7. A medium according to claim 5, wherein determination of the difference for each of the plurality of candidate depths comprises determination of, over N samples above and N samples below, for each candidate depth i, and for each of a number of ultrasound beams M:
$${D}_{i} = {\sum }_{k = 1}^{M} {\sum }_{j = 0}^{N - 1} {B}_{k , i + N - j} - {\sum }_{k = 1}^{M} {\sum }_{j = 0}^{N - 1} {B}_{k , i - j}$$

8. A medium according to claim 7, wherein determination of the difference for each of the plurality of candidate depths comprises determination of the difference at depth i+1 from the difference at depth i as:
$$\begin{matrix}{D}_{i + 1} = {\sum }_{k = 1}^{M} {\sum }_{j = 0}^{N - 1} {B}_{k , i + 1 + N - j} - {\sum }_{k = 1}^{M} {\sum }_{j = 0}^{N - 1} {B}_{k , i + 1 - j} \\ = {\sum }_{j = 0}^{N - 1} {H}_{i + 1 + N - j} - {\sum }_{j = 0}^{N - 1} {H}_{i + 1 - j} \\ = {\sum }_{j = 0}^{N - 1} {H}_{i + N - j} - {H}_{i + 1} + {H}_{i + 1 + N} - {\sum }_{j = 0}^{N - 1} {H}_{i - j} + {H}_{i - N + 1} - {H}_{i + 1} \\ = {D}_{i} + {H}_{i - N + 1} - 2 \times {H}_{i + 1} + {H}_{i + 1 + N}\end{matrix}$$
where Dᵢ₊₁ is the difference determined for candidate depth i+1, and Hᵢ is the sum of samples across the beams at sample depth i:
$${H}_{i} = {\sum }_{k = 1}^{M} {B}_{k , i} .$$

9. An apparatus to:
receive a plurality of samples of an ultrasound beam from a transducer, each of the plurality of samples associated with a tissue depth;
for each of a plurality of candidate depths, determine a difference between the sum of each of the plurality of samples which is associated with a tissue depth from the candidate depth to a predetermined distance below the candidate depth, and the sum of each of the plurality of samples which is associated with a tissue depth from the candidate depth to the predetermined distance above the candidate depth;
determine the largest determined difference;
determine the one of the plurality of candidate depths associated with the largest determined difference;
calculating a time delay based on the candidate depth associated with the largest determined difference; and
transmitting a second ultrasound beam based on the calculated time delay.

10. An apparatus according to claim 9, wherein determination of the difference for each of the plurality of candidate depths comprises determination of, over N samples above and N samples below, for each candidate depth i:
$${D}_{i} = {\sum }_{j = 0}^{N - 1} {B}_{i + N - j} - {\sum }_{j = 0}^{N - 1} {B}_{i - j}$$
where Dᵢ is the difference determined for candidate depth i and Bᵢ is a beam sample at candidate depth i.

11. An apparatus according to claim 9, wherein determination of the difference for each of the plurality of candidate depths comprises determination of, over N samples above and N samples below, for each candidate depth i, and for each of a number of ultrasound beams M:
$${D}_{i} = {\sum }_{k = 1}^{M} {\sum }_{j = 0}^{N - 1} {B}_{k , i + N - j} - {\sum }_{k = 1}^{M} {\sum }_{j = 0}^{N - 1} {B}_{k , i - j}$$

12. An apparatus according to claim 11, wherein determination of the difference for each of the plurality of candidate depths comprises determination of the difference at depth i+1 from the difference at depth i as:
$$\begin{matrix}{D}_{i + 1} = {\sum }_{k = 1}^{M} {\sum }_{j = 0}^{N - 1} {B}_{k , i + 1 + N - j} - {\sum }_{k = 1}^{M} {\sum }_{j = 0}^{N - 1} {B}_{k , i + 1 - j} \\ = {\sum }_{j = 0}^{N - 1} {H}_{i + 1 + N - j} - {\sum }_{j = 0}^{N - 1} {H}_{i + 1 - j} \\ = {\sum }_{j = 0}^{N - 1} {H}_{i + N - j} - {H}_{i + 1} + {H}_{i + 1 + N} - {\sum }_{j = 0}^{N - 1} {H}_{i - j} + {H}_{i - N + 1} - {H}_{i + 1} \\ = {D}_{i} + {H}_{i - N + 1} - 2 \times {H}_{i + 1} + {H}_{i + 1 + N}\end{matrix}$$
where Dᵢ₊₁ is the difference determined for candidate depth i+1, and Hᵢ is the sum of samples across the beams at sample depth i:
$${H}_{i} = {\sum }_{k = 1}^{M} {B}_{k , i} .$$

13. An apparatus according to claim 11, wherein determination of the difference for each of the plurality of candidate depths comprises recursive determination of Sᵢ, where:
$$\begin{matrix}{S}_{i} = {\sum }_{k = 1}^{M} {\sum }_{j = 0}^{N - 1} {B}_{k , i - j} \\ = {\sum }_{j = 0}^{N - 1} {H}_{i - j} \\ = {\sum }_{j = 1}^{N} {H}_{i - j} - {H}_{i - N} + {H}_{i} \\ = {S}_{i - 1} - {H}_{i - N} + {H}_{i} ,\end{matrix}$$
where Hᵢ is the sum of samples across the beams at sample depth i:
$${H}_{i} = {\sum }_{k = 1}^{M} {B}_{k , i} , and$$
$$where {D}_{i} = {S}_{i + N} - {S}_{i} .$$
